(19) United States
(12) Patent Application Publication
(10) Pub. No.: US 2004/0064845 A1
(43) Pub. Date: Apr. 1, 2004

(54) Method of cloning animals

(76) Inventors: Lawrence Charles Smith, Saint-Hyacinthe (CA); Vilceu Bordignon, Saint-Hyacinthe (CA); Jose Henrique Fortes Pontes, Taubate(Centro) (BR)

Correspondence Address: OGILVY RENAULT, 1981 MCGILL COLLEGE AVENUE, SUITE 1600, MONTREAL, QC, H3A2Y3, CA

(21) Appl. No.: 10/432,611

(22) PCT Filed: Dec. 5, 2001
(86) PCT No.: PCT/CA01/01722

(51) Int. Cl. C12N 15/85

(52) U.S. Cl. 800/21; 435/455

(57) ABSTRACT

The present invention relates to cloning method of animals. The invention includes cell lines, reconstructed embryos and cloned or transgenic animals. In particular, the invention provides method of cloning animals by combining genome of donor cells at specific stages of the cell cycle without uses of chemical products, with activated enucleated oocyte to thereby obtain reconstructed embryos. The invention further relates to methods of culturing animal cells until confluence in normal conditions avoiding negative genetic mutations induced by chemicals. Also, the invention relates to a method of preparing recipient oocytes before nuclear transfer, and to a culture medium improving the in vitro as well as in vivo development of reconstructed embryos.

### BACKGROUND OF THE INVENTION

(a) Field of the Invention

The invention relates to a method of cloning animal cells and animals, by in vitro culture of genome donor cells, and introduction of the nucleus of a genome donor cell into functionally enucleated oocytes. The cell cycle stage of the cells is exploited to improve the cloning yield. The cells can be also genetically transformed prior to transfer into oocytes and allows for production of transgenic animals.

(b) Description of Prior Art

Researchers have been developing methods for cloning mammalian animals over the past two decades. These reported methods typically include the steps of (1) isolating a cell, most often an embryonic cell; (2) inserting the cell or nucleus isolated from the cell into an enucleated oocyte (e.g., the oocyte's nucleus was previously extracted), and (3) allowing the embryo to mature in vivo.

The first successful nuclear transfer experiment using mammalian cells was reported in 1983, where the pronuclei isolated from a murine (mouse) zygote were inserted into an enucleated oocyte and resulted in like offspring(s). McGrath & Solter, 1983, Science 220:13001302. Host oocytes are able to support better development after nuclear transfer when compared to pronuclear-enucleated host zygotes. It has already been shown that MII-stage enucleated oocytes either aged or activated before fusion support better development. The problem of using young non-activated oocytes is caused by incompatibilities between the cell cycle stages of the nuclear donor cell and the host cytoplasm. Metaphase arrested secondary (MII) oocytes have high levels of a Maturation Promoting Factor (MPF), a cellular activity that is responsible for maintaining the chromatin condensed without a nuclear envelop. When blastomere interphase-stage nuclei containing decondensed chromatin are introduced into an MII oocyte, MPF leads to a rapid breakdown of the nuclear membrane and premature chromosome condensation (PCC). However, PCC is believed to be detrimental only when induced during the DNA synthesis stage (S-phase) of cell cycle. This is particularly problematic when using donor nuclei from blastomeres since these undergo S-phase for most time in between cell divisions. On the other hand, enucleated oocytes that have been activated or aged before fusion to nuclear donor cells have lower levels of MPF and, therefore, do not cause PCC.

Current knowledge indicates that the only way to succeed with cloning is to use a G0/G1 nucleus with metaphase stage enucleated oocytes. It was demonstrated that telophase stage enucleated oocytes could be successfully used with non-synchronous nuclei. It was shown that nuclear donor cells synchronized at S or G2/M stage are significantly better then those G1-phase cells when using telophase-enucleated host oocytes.

Once the nuclear donor cell has been introduced into the perivitelline space of the enucleated oocyte, the couplet is exposed to a DC electric pulse that causes fusion between the plasma membranes leading to the entry of the donor nucleus into the enucleated oocyte. Apart from introducing the donor nucleus, the reconstructed oocyte must be activated to initiate development. Although oocyte activation is usually triggered by the entry of the sperm at fertilization, activation can also be induced in the absence of a sperm (parthenogenesis) by exposing the oocyte to different stimuli, i.e. temperature shock, electric shock, calcium ionophore, etc. However, oocytes do not always remain “activated” after an artificial stimulation. The most common strategies that have been used to ascertain development after activation is to expose the stimulated oocyte to inhibitors of either phosphorylation, i.e., 6-dimethylaminopurine (6-DMAP), or protein synthesis, i.e., cycloheximide. However, since studies have shown major chromosomal abnormalities in parthenogenetic embryos exposed to such agents, these products should be avoided during the activation of nuclear transfer embryos for cloning. Moreover, the present studies have indicated that the use of these inhibitors blocks or delays the remodeling of somatic histone H1 on donor chromatin.

With the exception of blastomeres, most other cell types have longer gaps both before (G1-phase) and after (G2-phase) the S-phase and, therefore, are less susceptible to the harmful effects of S-phase PCC when fused to a MII oocytes. Because high MPF levels cause the breakdown of the nuclear membrane, MII stage host oocytes are believed to facilitate interactions between donor nuclei and putative oocyte cytoplasmic ‘factors’ required for reprogramming the chromatin of nuclei derived from cells further advanced in differentiation. Several examples in the literature report on the advantages of passing further differentiated donor nuclei in non-activated MII oocytes before activating the reconstructed oocyte. In cattle, nuclei from an embryonic cell line supported significantly higher yield of blastocyst development and more 30 d pregnancies when fused to enucleated oocytes 4 h before activation. In mice, significantly more embryos reconstructed with cumulus cell nuclei developed to the blastocyst stage by exposing the donor nucleus to MII cytoplasm for between 1 and 6 h before activation.

Current knowledge indicates that the best way to prepare nuclear donor cells for cloning is to expose them to medium containing reduced amounts of serum (serum starvation). Exposure to serum-starvation conditions usually lasts between 5 to 10 days. The argument behind this procedure is that the removal of essential nutrients from the medium leads the cells to exit the cell cycle and arrest at a specific stage known as G0 (G-zero or G-naught). The first advantage of G0 cells is that the cells are not undergoing DNA replication (i.e., quiescent) at the time of fusion to the host oocyte. Second, since cells are mostly moribund due to lack of nutrients, the chromatin may be less restricted by packaging proteins that regulate gene expression and, therefore, more amenable to reprogramming. This technology has been used to produce clones from fetal and adult cells in different species. To counteract the need of serum-starvation for succeeding with cloning, others have used the cloning procedure with non-quiescent (cycling) cells.

It is well known that the stage of maturation of the oocyte at enucleation and nuclear transfer is important. In general, successful mammalian embryonic cell cloning practices use the metaphase II stage oocyte as the recipient oocyte. At this stage, it is believed the oocyte is sufficiently “receptive” to activation to treat the introduced nucleus as it does a fertilizing sperm.

The steps to activate mammalian oocytes involve generally 1) exit from meiosis; 2) reentry into the mitotic cell cycle by the secondary oocyte; and 3) the formation and migration of pronuclei within the cell. Competent oocytes prepared for maturation and subsequent activation are required for nuclear transfer techniques.

A transition is necessary for activation of oocytes. Among others, a Maturation Promoting, Factor complex becomes essential in the understanding of oocyte senescence and age dependent responsiveness to activation. MPF activity is partly a function of calcium. A major imbalance in the components of the multi-molecular complex which is required for cell cycle arrest may be responsible for the increasing sensitivity of oocytes to activation stimuli during aging.

Serum is often added to in vitro culture systems as a source of the necessary nutrients and growth factors that lack in balanced salt solutions. However, because of its unknown and variable composition, the use of serum in culture media during the early stage of embryo development has been directly related with abnormal growth patterns in both cattle and sheep. Therefore, development of chemically defined in vitro culture systems that lack serum are of great interest for many embryo biotechnologies that require exposure to in vitro environments, including mammalian adult cloning. Apart from the correct balancing of minerals in culture media, energy and amino acid composition, and the concentration of oxygen in the atmosphere seem to play an important role in supporting early development.

Despite the progress of cloning ovine and bovine animals, there remains a great need in the art for methods and materials that increase cloning efficiency. In addition there remains a great need in the art to expand the variety of cells that can be utilized as nuclear donors, especially expanding nuclear donors to non-embryonic cells. Furthermore, there remains a long felt need in the art for karyotypically stable permanent cell lines that can be used for genome manipulation and production of transgenic cloned animals.

For successful commercial use of techniques such as genetic engineering or cloning, it must be possible to mature a single-cell embryo in vitro to the morula or blastocyst stage before it can be non-surgically transferred into a surrogate recipient dam to produce a pregnancy. However, embryos from different species, as bovine, encounter a block to in vitro bovine embryonic development at the 8- to 16-cell stage. Numerous efforts have been made to overcome this block to in vitro embryo development.

Eyestone, et al., (1987, Theriogenology 28:1-7) reported that ligated ovine oviducts would support development of bovine embryos from the 1-cell to blastocyst stage. Pregnancies and live calves were produced after transfer of cultured embryos to recipient heifers. Cultures of 1- and 2-cell embryos in the oviducts of intact cycling, ovariectomized and anestrous ewes produced morphologically normal morulae and blastocysts followed by pregnancies in recipient heifers, suggesting that ovarian activity was not required for normal embryo development in the oviduct.

These results suggest that there is a stage in bovine embryonic development, perhaps the 5- to 8-cell stage, which is a period of particular sensitivity to in vitro conditions, Therefore, it is likely that an important, environmentally sensitive event occurs around the 8-cell stage of embryonic development. Exposure of embryos to suboptimal conditions during this period may prevent the normal occurrence of this event, thus blocking further development.

Moreover, it would be highly desirable to be provided with a new method of cloning that eliminates the possible adverse effects of long term nutrient depletion on chromatin integrity, as chromatin fragmentation is known to occur during the first stages of apoptosis and cell death, with a new method of preparing oocytes to accomplish the nuclear transfer necessary for cloning, and with a new culture medium of embryos which would improve the hatching rate, in vitro development yield as well as the in vivo development.

### SUMMARY OF THE INVENTION

One object of the present invention is to provide a new method of cloning that eliminates possible adverse effects of long term nutrient depletion on chromatin integrity.

Another object of the present invention is to provide a method of cloning in which genomes from cells rendered at the G1-phase of the cell cycle by in vitro culture of the cells until confluence are introduced into functionally enucleated oocytes.

One object of the present invention is to provide method of preparing genome donor animal cells for cloning animals comprising the steps of:

a) culturing animal cells for a period of time sufficient to allow said cells to reach confluence and/or G1-phase of the cell cycle; and

b) isolating whole cell and/or genome of the cultured cells of step a) to obtain a genome donor cell.

In accordance with the present invention, there is provided a method of preparing genome donor animal cells with a further step of culturing G1-phase cells to reach the S or G2/M-phase of the cell cycle.

Another object of the present invention is to provide method of cloning an animal with a cell at G1-phase of the cell cycle comprising the steps of:

a) culturing animal cells for a period of time sufficient to allow the cells to reach confluence and G1-phase of the cell cycle;

b) introducing the whole cell and/or genome of the cultured cells of step a) into enucleated oocyte to obtain reconstructed embryos; and

c) developing the reconstructed embryo of step b) to obtain an animal.

The method according to the invention may comprise a further step after step a) of culturing the G1-phase cells to reach the S or G2/M phase of the cell cycle. In all variations of the present invention, the cells may be arrested at the S or G2/M stage of the cell cycle by submitting it to inhibitors in the culture medium before to reach these cell stages.

The culture of embryos may be performed in vitro.

The method according to the invention may further comprise implanting the reconstructed embryos of step b) into a surrogate mother and allowing the implanted embryo to develop into an animal.

Also, the genome donor cells of the invention may be selected from the group consisting of somatic cells, germ cells, embryonic cells, and stem cells. The cells may be transgenic cells, genetically transformed cells, transfected cells, and infected cells. The cells of the invention may be also selected from the group consisting of embryonic cells, foetal cells, fibroblast cells, epithelial cells, neural cells, keratinocytes, epidermal cells, hematopoietic cells, melanocytes, chondrocytes, lymphocytes, erythrocytes, muscle cells, and nuclei isolated therefrom. The cells of the invention may be provided by mammals, birds, reptiles, fishes, bovine, porcine, equine, canine, feline, ovine, caprine, primate, or any transgenic animal thereof.

In accordance with the invention, enucleated oocyte may be in a stage of a meiotic cell cycle selected from the group consisting of metaphase I, metaphase II, anaphase I, anaphase II, and telophase II.

The oocyte of the invention may be chemically, biochemically, biologically, enzymatically, and/or physically activated after enucleation.

The chemical activation may be performed by treatment with ethanol, ionophore, or ionomycin activation, and physical activation by electrical, thermal, and irradiation treatment.

In accordance with the invention, there is provided a method of preparing genome donor cells using oocyte that may be functionally enucleated by chemical, biochemical or enzymatic inactivation of the genome, or by X-ray irradiation, by laser irradiation, or by physical removal. Enucleated may be carried out in a medium comprising cytoskeletal inhibitors.

Another object of the invention is to provide a method of activating an oocyte for cloning animals comprising the steps of:

a) enucleating maturing oocyte between 18 to 26 hours of maturation and allowing the enucleated oocyte to mature for an additional period of time between 2 to 10 hours, or enucleating an oocyte between 26 to 34 hours of maturation; and

b) activating the enucleated oocyte of step a) before and/or after having transferred a donor cell into the oocyte.

In accordance with the present invention, oocyte of step a) may be physically, chemically, or functional enucleated. Electrical means, thermal means, irradiation technology, and/or chemical means may activate the oocytes of the invention.

Another object of the invention is to provide a composition for culturing embryos in vitro comprising modified glucose and/or glycine and alanine, wherein the modified glucose is at concentration between about 0 to 1.5 mM, the glycine is at concentration between about 1.0 to 2.0 mM, the alanine at concentration between about 0.5 to 1.0 mM.

Also, another object of the invention is to provide a method of cloning an animal comprising the steps of:

a) culturing animal cells for a period of time sufficient to allow the cells to reach confluence and G1-phase of cell cycle, or further to reach the S or G2/M phase of cell cycle;

b) enucleating maturing oocyte between 18 to 26 hours of maturation and allowing the enucleated oocyte to mature for an additional period of time between 2 to 10 hours, or enucleating an oocyte between 26 to 34 hours of maturation;

c) introducing a whole cell and/or genome of the cultured cells of step a) into the enucleated oocyte of step b) to obtain reconstructed embryos, wherein the enucleated oocyte of step a) is inactivated before and/or after introduction of the cell and/or genome the cell into the oocyte;

d) developing the reconstructed embryo of step c.) to obtain an animal.

The reconstructed embryos may be then implanted into a surrogate mother and allowed to develop into an animal.

The G1-phase cells may be treated with an inhibitor to arrest at the S or G2/M phase of the cell cycle.

In accordance with the method of cloning of the present invention, the reconstructed embryos may be cultured in in vitro conditions in a culture medium comprising modified glucose and/or glycine and alanine before implantation into surrogate mother to develop into an animal.

For the purpose of the present invention the following terms are defined below.

The term “confluence” as used herein is intended to mean a group of cells where a large percentage of the cells are physically contacted with at least one other cell in that group. Confluence may also be defined as a group of cells that grow to a maximum cell density in the conditions provided. For example, if a group of cells can proliferate in a monolayer and they are placed in a culture vessel in a suitable growth medium, they are confluent when the monolayer has spread across a significant surface area of the culture vessel. The surface area covered by the cells preferably represents about 50% of the total surface area, more preferably represents about 70% of the total surface area; and most preferably represents about 90% of the total surface area. The cultured cells can be organized at confluence in mutilayers.

The term “monolayer” is intended to mean cells that are attached to a solid support while proliferating in suitable culture conditions. A small portion of the cells proliferating in the monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support. Cells can also grow in culture in multilayers. The term “multilayers” as used herein refers to cells proliferating in suitable culture conditions where at least 15% of the cells are indirectly attached to the solid support through an attachment to other cells. Preferably, at least 25% of the cells are indirectly attached to the solid support, more preferably at least 50% of the cells are indirectly attached to the solid support, and most preferably at least 75% of the cells are indirectly attached to the solid support.

The term “oocyte,” as used here for the recipient oocyte, means an oocyte which develops from an oogonium and, following meiosis, becomes a mature ovum. It has been found that not all oocytes are equally optimal cells for efficient nuclear transplantation in mammals. For purposes of the present invention, metaphase II stage oocytes, matured either in vivo or in vitro, have been found to be optimal. Mature metaphase II oocytes may be collected surgically from either nonsuperovulated or superovulated cows or heifers 24-48 hours past the onset of estrus or past an injection of human Chorionic Gonadotrophin (hCG) or similar hormone. Alternatively, immature oocytes may be recovered by aspiration from ovarian follicles obtained from slaughtered cows or heifers and then may be matured in vitro in a maturation medium by appropriate hormonal treatment and culturing. As stated above, the oocyte is allowed to mature in a known maturation medium until the oocyte enters the metaphase II stage, generally 24 to 34 hours post aspiration. For purposes of the present invention, this period of time is known as the “maturation period.”

The term “somatic cell” as used herein is intended to mean a cell that is not isolated from an embryo. Non-embryonic cells can be differentiated or non-differentiated. Non-embryonic cells can refer to nearly any somatic cell, such as cells isolated from an ex utero animal. These examples are not meant to be limiting.

The term “embryonic stem cell” as used herein is intended to mean pluripotent cells isolated from an embryo that are maintained in in vitro cell culture. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells are well known to a person of ordinary skill in the art.

The term “nuclear transfer” as used herein is intended to mean introducing a full complement of nuclear DNA from one cell to an enucleated cell. Nuclear transfer methods are well known to a person of ordinary skill in the art. See, U.S. Pat. No. 4,994,384, entitled “Multiplying Bovine Embryos,” Prather et al., issued on Feb. 19, 1991 and U.S. Pat. No. 5,057,420, entitled “Bovine Nuclear Transplantation,” Massey, issued on Oct. 15, 1991, both of which are hereby incorporated by reference in their entirety including all figures, tables and drawings. Nuclear transfer may be accomplished by using oocytes that are not surrounded by a zona pellucida.

The term “modified glucose” as used herein is intended to mean that the glucose concentration may be reduced at a minimal level or absent to the culture medium. Modified glucose may also be a derivative of the native glucose, or a chemically modified form.

This summary of the invention does not necessarily describe all necessary features of the invention, but that the invention may also reside in a sub-combination of these described features. The summary of the invention, thus incorporated, presents, therefore, only an example but not a limitation of subject matter to exactly this combination of features.

### DETAILED DESCRIPTION OF THE INVENTION

The method in accordance with the present invention is different from both previous procedures because it uses a system where donor cells are synchronized at the G1-phase (before DNA synthesis) by confluence. As cell replenish the surface area in the culture dish, i.e. become confluent, they arrest their cycling activity due close contact with neighboring cells, i.e., contact inhibition. The following experiments indicate that 95% of the cells arrest at G1 after achieving confluence. This is at least as good as the level of G0/G1 synchronization obtained by serum-starvation.

The method of cell synchronization at the S or G2/M phase of the present invention may comprise: (1) allowing nuclear donor cells to grow to confluence; (2) remove G1-synchronized (confluent) donor cells from a dish and plating at low density in a new dish; (3) allow cells to reinitiate the cell cycle and arrive at the S or G2/M phase after a short period of time; and (4) use cell cycle inhibitors to arrest or block entry into mitosis. Donor cells are then used in nuclear transfer using telophase-enucleated oocytes.

According to another embodiment of the present invention, the cells may be arrested to the S or G2/M stage of the cell cycle by submitting it to an inhibitor that is added to the culture medium. One example of such an inhibitor is the roscovitine.

In one embodiment of the present invention, nuclear transfer may comprise: (1) use of oocytes that are enucleated at approximately 24 h of maturation and returned to maturation drop for a further 4 to 6 h before nuclear transfer; (2) At 28 to 30 h after the beginning of maturation, enucleated oocytes are manipulated to introduce the donor cell into the perivitelline space; (3) manipulated oocytes are placed into a electrofusion solution, aligned and exposed to a DC electric current; (4) nuclear transfer oocytes that have fused are exposed to a solution containing a calcium ionophore (ionomycin) for a short period to induce activation; (5) after exposure to ionomycin, nuclear transfer oocytes are transferred to embryo culture medium without inhibitors of cell cycle kinases or protein synthesis.

Also, oocytes of the invention may be enucleated at 28 to 34 hours of maturation, prior to be used for nuclear transfer. The use of aged mature oocytes in nuclear transfer procedures avoids the return of MPF activity to its initial high concentration, which is deleterious for reconstructed oocytes and embryos in this context, that is the case in techniques using inhibitors of phosphorylation and protein synthesis to perform the same step.

The present invention allows nuclear transfer processes to proceed with older oocytes such as a 30-hour oocyte, which may produce healthier embryonic cells, superior blastocyst developmental and hatching rates. There is evidence indicating that late oocyte activation allows for better development of the nuclear transplanted cell. The 30-hour oocyte is the approximate age at which the concentration of MPF will not go back after nuclear transfer.

One embodiment of the invention is a culture medium that is capable of supporting development to blastocysts and blastocyst hatching. The developmental rates are superior to other known culture medium and systems. It has been used to culture embryos cloned from adult cells leading to the birth of a calf showing no abnormalities.

Another embodiment of the present invention provides with a method of cloning animals by combining a preparation of donor cells by confluence synchronization, transfer these donor cells in activated enucleated oocytes according to the invention, and developing resulting reconstructed oocytes and embryos in the culture medium according to the present invention before transfer into a recipient mother.

The term “modified nuclear DNA” as used herein refers to the nuclear deoxyribonucleic acid sequence of a cell, embryo, fetus, or animal of the invention that has been manipulated by one or more recombinant DNA techniques. Examples of these recombinant DNA techniques are well known to a person of ordinary skill in the art, which can include (1) inserting a DNA sequence from another organism (e.g., a human organism) into target nuclear DNA, (2) deleting one or more DNA sequences from target nuclear DNA, and (3) introducing one or more base mutations (e.g., site-directed mutations) into target nuclear DNA. Cells with modified nuclear DNA can be referred to as “transgenic cells” for the purposes of the invention. Transgenic cells can be useful as materials for nuclear transfer cloning techniques provided herein.

Methods and tools for insertion, deletion, and mutation of nuclear DNA of mammalian cells are well known to a person of ordinary skill in the art. See, Molecular Cloning, a Laboratory Manual, 2nd Ed., 1989, Sambrook, Fritsch, and Maniatis, Cold Spring Harbor Laboratory Press; U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos,” all of which are incorporated by reference herein in their entirety, including all figures, drawings, and tables. These methods include techniques for transfecting cells with foreign DNA fragments and the proper design of the foreign DNA fragments such that they effect insertion, deletion, and/or mutation of the target DNA genome.

One particular embodiment of the present invention is the cloning of transgenic cells. Transgenic cells, including genetically modified cells, transfected cells, or infected cells, may be obtained in a variety of manners. For example, transgenic cells can be isolated from a transgenic animal. Examples of transgenic animals are well known in the art, as described herein with regard to transgenic bovine and ovine animals. Cells isolated from a transgenic animal can be converted into totipotent and/or immortalized cells by using the materials and methods provided herein. In another example, transgenic cells can be created from totipotent and/or immortalized cells of the invention. Materials and methods for converting non-transgenic cells into transgenic cells are well known in the art, as described previously. The transgenic cells may then be used in cloning protocols to produce transgenic animals.

Any of the cell types defined herein can be altered to harbor modified nuclear DNA. For example, embryonic stem cells, cells from the inner cell mass of young embryos, fetal cells, and any totipotent and immortalized cell defined herein can be altered to harbor modified nuclear DNA.

Examples of methods for modifying a target DNA genome by insertion, deletion, and/or mutation are retroviral insertion, artificial chromosome techniques, gene insertion, random insertion with tissue specific promoters, homologous recombination, gene targeting, transposable elements, and/or any other method for introducing foreign DNA. Other modification techniques well known to a person of ordinary skill in the art include deleting DNA sequences from a genome, and/or altering nuclear DNA sequences. Examples of techniques for altering nuclear DNA sequences are site-directed mutagenesis and polymerase chain reaction procedures. Therefore, the invention provides for animal cells that are simultaneously totipotent, immortalized, and transgenic. These transgenic, totipotent, immortalized cells can serve as nearly unlimited sources of donor cells for production of cloned transgenic animals.

The present invention has application in the genetic transformation of multicellular eukaryotic organisms by a new cloning approach. Examples of such organisms include amphibians, reptiles, birds, and mammal. In another embodiment of the invention, there is provided with a reliable in vitro culture medium that allows the development of early bovine embryos to blastocyst stage. Such a development may replace the surrogate oviduct system by an in vitro culture system and would greatly facilitate embryo manipulation procedures. The lack of a reliable in vitro culture system for early bovine embryos has hampered studies of early development and the application of these manipulation procedures. The culture medium of the present invention allows also particularly producing reconstructed embryos having improved capacities of hatching, in vitro and in vivo development.

In a particular embodiment of the invention, there is provided an embryo culture medium allowing in vitro development without the requirement for serum, specifically fetal calf serum, or the use of a co-culture of animal cells or other biological media, i.e., media comprising animal cells (e.g. epithelial cells) or other complex proteins.

In one embodiment, the present invention advantageously comprises a simple composition. As with most culture media known to the art, the culture medium includes a culture solution containing substances that are nutritionally necessary to support the embryos. Advantageously, the simple embryo culture medium of the present invention is formed without the requirement for fetal calf serum and glucose.

Of particular embodiment, the culture medium of the invention comprises products, and adjustment of amino acid contents With addition of specific concentrations of glycine and alanine.

According to one embodiment, the present invention is particularly useful in the production of animals of agriculture value, to obtain species having a genetic makeup that results in an animal having more desirable characteristics.

Method of Preparing Nuclear Donor Cells for Cloning at G1-Phase of the Cell Cycle (G1-Phase Donor Cells used with Metaphase Oocytes)

Fetal or adult skin-derived fibroblasts were obtained from tissue biopsies and cultured in DMEM™ medium supplemented with 10% FCS. Proliferating cells were passed once and aliquoted for freezing at a second passage. Frozen cells were thawed and plated at 10,000 cells/ml in plastic culture dishes with 6-cm diameter. After 3 days of culture cells reached confluence and were used for nuclear transfer 2-4 days after attaining confluence. Flow cytometry analysis showed that approximately 95% (96-98% for fibroblasts and 93-96% for granulosa cells) of the cells are at the G1/G0-phase at 48 h of culture in confluence. The developmental potential of embryos produced by nuclear transfer was compared between cells synchronized by confluence and those synchronized by serum starvation (5 days of culture in DMEM™ medium supplemented with 0.5% of FCS). Development to blastocyst stage after 7 days in culture was similar between cells synchronized by confluence and serum starvation when using fetal (19 vs. 22%) and adult (26 vs. 27%) fibroblasts.

Metaphase-Stage Oocyte Enucleation

Follicles with 2 to 8 mm diameter were aspirated from bovine slaughterhouse ovaries. Oocytes with a homogeneous cytoplasm and several layers of cumulus cells were selected and placed in maturation medium. At 22 h after maturation oocytes were denuded of cumulus cells and those with a first polarbody were used in the experiment. Selected oocytes were placed in medium containing cytochalasin B (5 μg/ml; micromanipulation medium) and the first polarbody and the surrounding cytoplasm were aspirated. Exposure to a vital dye (Hoechst 33342) and ultraviolet light indicate that 60 to 70% of the oocytes did not contain meiotic chromosomes, i.e., were enucleated, after the aspiration procedure. Enucleated oocytes are returned to maturation medium for a further 6 h until nuclear transfer. After this period, a single donor cell was introduced into the perivitelline space and electrofused by exposure to a 1.5 KV/cm electric pulse lasting 70 psec. After electrical stimulation, oocytes are washed, placed cultured medium for another 1-2 h and examined for fusion. Fused couplets derived from metaphase-stage enucleated oocytes were placed in medium containing 5 μM ionomycin to induce activation. No inhibitors of protein synthesis or kinase activity were used after activation with ionomycin.

Method of Preparing Nuclear Donor Cells for Cloning at G2/M-Phase of the Cell Cycle (G2/M-Phase Donor Cells used with Telophase Oocytes)

Confluent donor cells were plated at 10,000-20,000 cells/ml in DMEM medium with 10% of FCS and cultured for 16 to 24 h before use in nuclear transfer. Flow cytometry assessment indicated that 45-75% of cells was at S phase at 16 h and 20-55% was at G2-M phase at 24 h after plating. Nuclear transfer was performed with cells at 16 to 24 h post plating and development to blastocyst stage were 24% using preactivated telophase-II enucleated oocytes compared with 11% for M-II enucleated oocytes. Inhibiting entry into mitosis with specific (roscovitine) or non-specific (6DMAP) kinase inhibitors can increase the percentage of cells at G2/M-phase.

Telophase-Stage Enucleated Oocytes

Follicles with 2 to 8 mm diameter were aspirated from bovine slaughterhouse ovaries. Oocytes with a homogeneous cytoplasm and several layers of cumulus cells were selected and placed in maturation medium. At 28 h after maturation oocytes were denuded of cumulus cells and those with a first polarbody were used in the experiment. Oocytes were exposed to 5-μM ionomycin and cultured for a further 2 h. Oocytes with expelling or expelled second polarbodies were enucleated at telophase II-stage by removing approximately one-tenth of the cytoplasm adjacent to the second polar body. Nuclear donor cells were injected into the perivitelline space and fused to the telophase-enucleated host cytoplast at approximately 2.5-h after activation.

Chemically Defined Medium for Culturing, Embryos In Vitro

Embryo Culture Medium

Serum is often added to in vitro culture systems as a source of the necessary nutrients and growth factors that lack in balanced salt solutions. However, because of its unknown and variable composition, the use of serum in culture media during the early stage of embryo development has been directly related with abnormal growth patterns in both cattle and sheep. Therefore, development of chemically defined in vitro culture systems that lack serum are of great interest for many embryo biotechnologies that require exposure to in vitro environments, including mammalian adult cloning. Apart from the correct balancing of minerals in culture media, energy and amino acid composition, and the concentration of oxygen in the atmosphere seem to play an important role in supporting early development.

Experiments to Test Embryo Culture Medium

All cultures used tested using in vitro matured and fertilized bovine zygotes (presumptive-zygotes) and were performed in 50 μl drops of medium under equilibrated mineral oil in 5% CO₂ at 38° C.

Experiment 1: Effects of Glucose on Development to the Blastocyst Stage

The control in vitro culture group was based on Menezo B2™ culture medium supplemented with 10% FCS in the presence of bovine oviductal cells at atmospheric (18%) oxygen levels. Our treatment groups were based on SOF medium modified by supplementing with 8 mg/ml of fatty acid-free BSA and 1 mM glutamine cultured in 5% oxygen. Treatment 1 contained 0.5-mM glucose and treatment 2 contained 1.5-mM glucose. The percentage development to the blastocyst stage was superior in 0.5-mM glucose medium (33%) when compared to 1.5 glucose (26%) and control (23%) media. These results indicate that lower levels of glucose (0.5 mM) support better in vitro development to the blastocyst stage.

Experiment 2: Effects of Alanine and Glycine at Oviductal Concentrations

Based on results from Experiment 1, the control in vitro culture group was based on the modified SOF medium containing with 0.5-mM glucose. In an attempt to simulate the amino acid concentrations present in the oviduct a treatment group was supplemented with 0.5-mM alanine and 1.5 mM glycine. Although no significant difference in blastocyst stage development was obtained at day 7 of culture (38 vs. 41%), significantly more blastocysts hatched from the zona pellucida at day 9 when cultured with extra alanine and glycine than controls (75% vs. 47%). These results indicate that alanine and glycine at oviductal concentrations support better long-term development during culture in vitro, suggesting that embryos may produce higher gestation rates after transfer into the uteri of recipient females. The latter is supported by the production of a healthy somatic cell cloned calf derived using the above in vitro culture medium:

Method Used to Produce Calves by Somatic Cell Cloning

Method 1: Confluent Donor Cells with Metaphase-Arrested Host Oocytes

a) fibroblasts from the skin of a day 55 fetus are plated at 10⁶ cell/ml in a 60 mm diameter dish in medium alpha-DMEM supplemented with 10% of fetal calf serum;

b)fibroblasts are cultured for 4 days at 38° C. until cell cycle arrest by confluence inhibition (mostly at G1/G0 stage of the cell cycle);

c)confluent-arrested cells are trypsinized and used within one hour in nuclear transplantation experiments;

d)host oocytes were enucleated at metaphase-stage (M-II) at 22 h from the beginning of in vitro maturation (IVM), fused to at 26 h and activated at 28 h after IVM;

f) confluence-arrested fibroblasts were positioned within the perivitelline space of enucleated M-II oocytes and exposed to an electric current for fusion at 26 h after IVM;

g) at 28 h after IVM, reconstructed (fused) oocytes were exposed to 5 μM Ionomycin in TCM-199 hepes-buffered medium during 4 minutes;

h) reconstructed oocytes were cultured for 8 days in CRRA-modified SOF medium at 38.5° C. in an atmosphere of 5% CO₂ and 5% O₂.

i) blastocyst-stage embryos were transferred to synchronized recipient heifers and allowed to develop to term.

Method 2: Roscovitine-Arrested Donor Cells with Telophase-Enucleated Host Oocytes

a) confluent-arrested fibroblasts (Method 1) were plated into dishes at low density and cultured for 20 h to enable initiation of cycling activity (most cells are in the S-phase of the cell cycle);

b) cycling cells exposed to roscovitine at 50 AM for 8 h, at which stage most cells are arrested at the G2/M phase of the cell cycle;

c) host oocytes were activated with ionomycin (as described in Method 1) at 28 h after IVM and enucleated and fused to roscovitine-arrested donor cells 2.5 h later;

d) reconstructed oocytes were cultured for 8 days in CRRA-modified SOF medium at 38.5° C. in an atmosphere of 5% CO₂ and 5% O₂.

e) blastocyst-stage embryos were transferred to synchronized recipient heifers and allowed to develop to term.

Results

TABLE 1
Preliminary results comparing the gestation outcome of
embryos reconstructed using methods 1 and 2.

| Method of | Recipients | Gestations (%) | | |
|---|---|---|---|---|
| Reconstruction | Transferred | Day 30 | Day 60 | Day 250 |
| Method 1 | 5 | 3 (60%) | 2 (40%) | 1 (20%) |
| Method 2 | 5 | 3 (60%) | 3 (60%) | 2 (40%) |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

What is claimed is:

1. A method of preparing genome donor animal cells for cloning animals comprising the steps of:
a)culturing animal cells for a period of time sufficient to allow said cells to reach confluence and/or G1-phase of the cell cycle; and
b)isolating whole cell and/or genome of said cultured cells of step a) to obtain a genome donor cell.

2. The method of claim 1, which further comprises a step i) after step a), culturing GI-phase cells to reach the S or G2/M-phase of the cell cycle.

3. A method of cloning an animal with a cell at G1-phase of the cell cycle comprising the steps of:
a)culturing animal cells for a period of time sufficient to allow said cells to reach confluence and G1-phase of the cell cycle;
b)introducing the whole cell and/or genome of said cultured cells of step a) into enucleated oocyte to obtain reconstructed embryos; and
c)developing said reconstructed embryo of step b) to obtain an animal.

4. The method according to claim 3, which further comprises a step i) after step a), culturing said G1-phase cells to reach the S or G2/M phase of the cell cycle.

5. The method according to claim 4, wherein said G1-phase cells are treated with an inhibitor to arrest at the S or G2/M phase of the cell cycle.

6. The method according to claim 3, which further comprises culturing said reconstructed embryos of step b) in in vitro condition.

7. The method according to claim 3, which further comprises implanting said reconstructed embryos of step b) into a surrogate mother and allowing said implanted embryo to develop into an animal.

8. The method according to claim 1 or 3, wherein said cell is selected from the group consisting of somatic cells, germ cells, embryonic cells, and stem cells.

9. The method according to claim 1 or 3, wherein said cell are selected from the group consisting of transgenic cells, genetically transformed cells, transfected cells, and infected cells.

10. An animal produced by the use of a cell as defined in claim 9.

11. The method according to claim 8, wherein said somatic cell is selected from the group consisting of embryonic cells, foetal cells, fibroblast cells, epithelial cells, neural cells, keratinocytes, epidermal cells, hematopoietic cells, melanocytes, chondrocytes, lymphocytes, erythrocytes, muscle cells, and nuclei isolated therefrom.

12. The method according to claim 1 or 3, wherein said enucleated oocyte is in a stage of a meiotic cell cycle selected from the group consisting of metaphase I, metaphase II, anaphase I, anaphase II, and telophase II.

13. The method according to claim 3, wherein said oocyte is activated after enucleation.

14. The method according to claim 13, wherein said oocyte is chemically, biochemically, biologically, enzymatically, and/or physically activated.

15. The method according to claim 14, wherein said chemical activation is ethanol, ionophore, or ionomycin activation.

16. The method according to claim 14, wherein said physical activation is selected from the group consisting of electrical, thermal, and irradiation activation.

17. The method according to claim 3, wherein said oocyte is functionally enucleated by chemical, biochemical or enzymatic inactivation of the genome, or by X-ray irradiation, by laser irradiation, or by physical removal.

18. The method according to claim 3, wherein said oocyte is enucleated in a medium comprising cytoskeletal inhibitors.

19. The method according to claim 1 or 3, wherein said animal is selected from the group consisting of mammals, birds, reptiles, and fishes.

20. The method according to claim 19, wherein said mammal is selected from the group consisting of bovine, porcine, equine, canine, feline, ovine, caprine, and primate.

21. The method according to claim 7, wherein said animal is a transgenic animal.

22. A method of activating an oocyte for cloning animals comprising the steps of:
a) enucleating maturing oocyte between 18 to 26 hours of maturation and allowing said enucleated oocyte to mature for an additional period of time between 2 to 10 hours, or enucleating an oocyte between 26 to 34 hours of maturation; and
b) activating said enucleated oocyte of step a) before and/or after having transfer a donor cell into said oocyte.

23. The method of claim 22, wherein said enucleation of said oocyte of step a) is selected from the group consisting of physical, chemical, and functional enucleation.

24. The method of claim 22, wherein said activation of step b) is performed by electrical means, thermal means, irradiation technology, and/or chemical means.

25. A composition for culturing embryos in vitro comprising modified glucose and/or glycine and alanine.

26. The composition of claim 25, wherein said modified glucose is at concentration between about 0 to 1.5 mM, said glycine is at concentration between about 1.0 to 2.0 mM, and said alanine at concentration between about 0.5 to 1.0 mM.

27. A method of cloning an animal comprising the steps of:
a) culturing animal cells for a period of time sufficient to allow said cells to reach confluence and G1-phase of cell cycle;
b) enucleating maturing oocyte between 18 to 26 hours of maturation and allowing said enucleated oocyte to mature for an additional period of time between 2 to 10 hours, or enucleating an oocyte between 26 to 34 hours of maturation;
c) introducing a whole cell and/or genome of said cultured cells of step a) into said enucleated oocyte of step b) to obtain reconstructed embryos, wherein said enucleated oocyte of step a) is inactivated before and/or after introduction of said cell and/or genome of said cell into said oocyte;
d) developing said reconstructed embryo of step c) to obtain an animal.

28. The method of claim 27, which further comprises a step i) after step a), culturing G1-phase cells to reach the S or G2/M phase of cell cycle.

29. The method according to claim 27, wherein said G1-phase cells are treated with an inhibitor to arrest at the S or G2/M phase of the cell cycle.

30. The method according to claim 27, which further comprises implanting said reconstructed embryos of step d) into a surrogate mother and allowing said implanted embryo to develop into an animal.

31. The method according to claim 27 or 30, which further comprises culturing said reconstructed embryos of step c) in in vitro condition in a culture medium comprising modified glucose and/or glycine and alanine before implantation into surrogate mother to develop into an animal.

32. The method according to claim 27, wherein said cell is selected from the group consisting of somatic cells, germ cells, embryonic cells, and stem cells.

33. The method according to claim 27, wherein said cell is selected from the group consisting of transgenic, genetically transformed, transfected, and infected cells.

34. The method according to claim 32, wherein said somatic cell is selected from the group consisting of embryonic, foetal, fibroblast, epithelial, neural, keratinocytes, epidermal, hematopoietic, melanocytes, chondrocytes, lymphocytes, erythrocytes, muscle cells, and nuclei isolated therefrom.

35. The method according to claim 27, wherein said enucleated oocyte is in a stage of a meiotic cell cycle selected from the group consisting of metaphase I, metaphase II, anaphase I, anaphase II, and telophase II.

36. The method according to claim 27, wherein said oocyte is activated after enucleation.

37. The method according to claim 36, wherein said oocyte is chemically, biochemically, biologically, enzymatically, and/or physically activated.

38. The method according to claim 37, wherein said chemical activation is ethanol, ionophore, or ionomycin activation.

39. The method according to claim 37, wherein said physical activation is selected from the group consisting of electrical, thermal, and irradiation activation.

40. The method of claim 27, wherein said enucleation of said oocyte of step a) is selected from the group consisting of physical, chemical, and functional enucleation.

41. The method according to claim 27, wherein said oocyte is functionally enucleated by chemical, biochemical or enzymatic inactivation of the genome, or by X-ray irradiation, by laser irradiation, or by physical removal.

42. The method according to claim 27, wherein said oocyte is enucleated in a medium comprising cytoskeletal inhibitors.

43. The method according to claim 27, wherein said animal is selected from the group consisting of a mammal, a bird, a reptile, and a fish.

44. The method according to claim 43, wherein said mammal is selected from the group consisting of a bovine, a porcine, a equine, a canine, a feline, a ovine, a caprine, and a primate.

45. The method according to claim 30, wherein said animal is a transgenic animal.

46. The method of claim 30, wherein said modified glucose is at concentration between about 0 to 1.5 mM, said glycine is at concentration between about 1.0 to 2.0 mM, and said alanine at concentration between about 0.5 to 1.0 mM.

47. A animal produced by the method as defined in claim 27.

48. A transgenic animal produced by the method as defined in claim 27.
